(19) United States
(12) Patent Application Publication
(10) Pub. No.: US 2011/0129552 A1
(43) Pub. Date: Jun. 2, 2011

(54) Acne Treatment

(75) Inventors: Amit Saha, Beaconsfield (AU); Steven Sher, Beaconsfield (AU)

(73) Assignee: Laderma Pty Ltd, Beaconsfield,NSW (AU)

(21) Appl. No.: 12/629,033

(22) Filed: Dec. 1, 2009

(51) Int. Cl. A61K 36/00; A61P 17/10

(52) U.S. Cl. 424/725

(57) ABSTRACT

A kit and method for a two-step acne treatment regimen. The kit comprises a wash composition and a cream treatment composition. The method comprising the steps of: using a wash composition to clean acne affected skin of an individual; and applying a cream treatment composition to the acne affected skin.

### FIELD OF THE INVENTION

The present invention relates to the treatment of scarring associated with acne.

The invention has been developed primarily as a composition and method for treatment of acne and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

Acne is a common inflammatory disease of human skin, and concentrates in skin areas where sebaceous glands are largest, most numerous, and/or most active. Mild forms of acne are evidenced by spotty irritations, whereby ordinary skin hygiene is a satisfactory treatment. However, inflammatory types of acne can result in pustules, infected cysts and, in extreme cases, canalizing inflamed and infected sacs appear. The effect of acne can become extensive and leave permanent, disfiguring scars.

Further, acne is very common in teenagers at puberty, at which time facial eruptions are known to cause such psychic trauma in many adolescents that they find it difficult to make personal adjustments. The sufferer may be constantly aware of the obvious facial blemishes. Providing a medicinal preparation and treatment which can minimise this effect can provide a definite benefit.

To reduce the severity of acne, various forms of medication have previously been topically applied to the skin.

Known topical acne treatment regimens typically include:
cleansing the skin of an individual afflicted with acne with an acne cleanser composition;
applying an acne spot treating composition to at least one lesion on the skin of the individual;
applying a hydrating day cream composition to the skin of the individual, the hydrating day cream composition; and
applying an acne night cream composition to the skin of the individual, the acne night cream composition.

A method for controlling acne which comprises thoroughly washing an acne affected skin area with water and a cleansing agent, rinsing the washed acne affected skin area to substantially remove any residual cleansing agent, drying the washed acne affected skin area, topically applying to the washed acne affected skin area an effective amount of a acne treatment composition, and permitting the acne treatment composition to remain on the acne affected skin area to allow absorption thereof into the sebaceous glands of the skin, requires significant effort and perseverance.

### OBJECT OF THE INVENTION

It is an object of the present invention to overcome or ameliorate at least one of the disadvantages of the prior art, or to provide a useful alternative.

It is an object of the invention in its preferred form to provide an improved composition and method for treatment of acne.

### SUMMARY OF THE INVENTION

According to a first aspect of the invention there is provided a kit for an acne treatment regime, the kit comprising:
a wash composition; and
a cream treatment composition.

According to a second aspect of the invention there is provided a method for an acne treatment regime, the method comprising the steps of:
a) using a wash composition to cleans acne affected skin of an individual; and
b) applying a cream treatment composition to the acne affected skin.

Preferably, the acne treatment regime is a two-step acne treatment regime. More preferably, the treatment regimen further assists in scar reduction.

Preferably, the wash composition comprises: Melaleuca Altemifolia (Tea Tree) Oil; and Hamamelis Virginiana (Witch Hazel) Distillate. More preferably, the wash composition comprises any one or more of the following: Lavandula Angustifolia (Lavender) Oil; and Calendula Officinalis (Calendula) Extract 1:2. Most preferably, the wash composition comprises any one or more of the following: Aloe Vera Inner Leaf Juice Powder 200:1; Sodium Chloride (Natrum Muriaticum) 12×; Silica 3×; and Palmarosa Essential Oil. The wash composition preferably comprises any one or more of the following: Disodium Cocoamphodiacetate; Caprylyl Glycol (and) Sorbic Acid (and) Phenoxyethanol; Benzyl Alcohol; PEG-150 Distearate; Polysorbate 20; Phenoxyethanol; Lactic Acid; Cocamidopropyl Betaine; Lauryl Glucoside; and Purified Water.

Preferably, the cream treatment composition comprises: Melaleuca Altemifolia (Tea Tree) Oil; Hamamelis Virginiana (Witch Hazel) Distillate; and Zinc PCA. More preferably, the cream treatment composition comprises any one or more of the following: Rosa Canina Fruit (Rose Hip) Oil; and Garcinia Mangostana (Mangosteen) Extract. Most preferably, the cream treatment composition comprises any one or more of the following: Calcarea Carbonica (Calcium Carbonate) 3× Pulsatilla Vulgaris 3×; Calendula Officinalis 6×; Lactic Acid; and Chamomilla Recutita (Chamomile) Extract. The cream treatment composition preferably comprises any one or more of the following: Glyceryl Stearate; Cetyl Palmitate (and) Sorbitan Palmitate (and) Sorbitan Olivate (Oliwax); Caprylyl Glycol (and) Sorbic Acid (and) Phenoxyethanol (Optiphen Plus); Hydroxyethyl Acrylate/Sodium Acryloyldimethyltaurate Copolymer (and) Squalane (and) Polysorbate 60 (Simulgel NS); Stearic Acid; Glycerine 99.5% (Vege); Capric/Caprylic Triglycerides; and Purified water.

Preferably, the wash composition comprises:
Sodium Chloride (Natrum Muriaticum) 12× at 0.050% w/w; Silica 3× at 0.050% w/w; Cocoamidopropyl Betaine at 4.000% w/w; PEG 150 Distearate at 4,000% w/w; Polysorbate 20 at 4.000% w/w; Di Sodium Cocoamphodiacetate at 2.000% w/w; Lauryl Glucoside at 2.000% w/w; Optiphen Plus (Caprylyl Glycol, Sorbic acid, Phenoxyethanol) at 1.5000% w/w; Witchazel Distillate at 1000% w/w; Calendula Officinalis Extract 1:2 at 0.500% w/w; Phenoxyethanol at 0.500% w/w; Benzyl Alcohol at 0.500% w/w; Lavender (Lavandula Angustifolia) Oil at 0.400% w/w; Tea Tree Oil at 0.400% w/w; Palmarosa Essential Oil at 0.200% w/w; Lactic Acid at 0.200% w/w; Aloe Vera Inner Leaf Juice Powder 200:1 at 0.050% w/w; and Purified Water to 100.000% w/w.

Preferably the cream treatment composition comprises:
Calendula Officinalis 6× at 1.000% w/w; Calcium Carbonate 3× at 0.500% w/w; Pulsatilla Vulgaris 3× at 0.500% w/w; Capric/Caprylic acid Triglycerides at 8.000% w/w; Oliwax LC at 4.000% w/w; Stearic Acid at 4.000% w/w; Lactic Acid at 2.000% w/w; Simugel NS (Hydroxyethyl Acrylate/Sodium Acryloydimethyltaurate copolymer, Squalene and Polysorbate 60) at 2.000% w/w; Witchazel Distillate at 2.000% w/w; Zinc PCA at 2.000% w/w; Sodium Hydroxide 46% solution at 1.700% w/w; Optiphen Plus (Caprylyl Glycol, Sorbic acid, Phenoxyethanol) at 1.500% w/w; Glycerine 99.5% (Vege) at 1.000% w/w; Rosehip oil at 1.000% w/w; Glyceryl Stearate at 1.000% w/w; Tea Tree Oil at 0.900% w/w; Chamomile Extract 1:1 at 0.500% w/w; Mangosteen Extract at 0.500% w/w; and Purified water to 100.000% w/w.

The treatment regimen preferably assists in scar reduction, and the cream treatment composition preferably includes: Rose hip oil; Lavender oil; Calendula oil; Evening primrose oil; Coconut oil; Avocado oil; Olive oil; Arnica oil; Hypericum oil; and Everlasting oil. Preferably, the cream treatment composition includes: substantially 0.5% Rose hip oil; substantially 0.5% Lavender oil; substantially 0.05% Calendula oil; substantially 0.2% Evening primrose oil; substantially 2% Coconut oil; substantially 2% Avocado oil; substantially 0.075% Olive oil; substantially 0.25% Arnica oil; substantially 0.15% Hypericum healing oil; and substantially 0.0025% Everlasting oil.

According to further aspect of the invention there is provided a wash composition as described herein.

Preferably the wash composition comprises: Melaleuca Alternifolia (Tea Tree) Oil; and Hamamelis Virginiana (Witch Hazel) Distillate. More preferably, the wash composition further comprises: Lavandula Angustifolia (Lavender) Oil; and Calendula Officinalis (Calendula) Extract 1:2. Most preferably, the wash composition further comprises: Aloe Vera Inner Leaf Juice Powder 200:1; Sodium Chloride (Natrum Muriaticum) 12×; Silica 3×; and Palmarosa Essential Oil.

According to further aspect of the invention there is provided a cream treatment composition as described herein.

Preferably, the cream treatment composition comprises: Melaleuca Alternifolia (Tea Tree) Oil; Hamamelis Virginiana (Witch Hazel) Distillate; and Zinc PCA. More preferably, the cream treatment composition further comprises: Rosa Canina Fruit (Rose Hip) Oil; and Garcinia Mangostana (Mangosteen) Extract. Most preferably, the cream treatment composition further comprises: Calcarea Carbonica (Calcium Carbonate) 3× Pulsatilla Vulgaris 3×; Calendula Officinalis 6×; Lactic Acid; and Chamomilla Recutita (Chamomile) Extract.

According to further aspect of the invention there is provided a kit for an acne treatment regime, substantially as herein described with reference to any one of the embodiments of the invention illustrated in the accompanying drawings and/or examples.

According to further aspect of the invention there is provided a method for an acne treatment regime, substantially as herein described with reference to any one of the embodiments of the invention illustrated in the accompanying drawings and/or examples.

### BRIEF DESCRIPTION OF THE DRAWINGS

A preferred embodiment of the invention will now be described, by way of example only, with reference to the accompanying drawings in which:

FIG. 1 shows an schematic diagram of an embodiment kit for an acne treatment regime; and

FIG. 2. shows an flowchart of an embodiment method for an acne treatment regime.

### PREFERRED EMBODIMENT OF THE INVENTION

It will be appreciated that embodiments of the present invention can be utilised in skin treatment, and in particular to the treatment of acne.

In an embodiment, by way of example only, this method can be used in an acne treatment regimen comprising a complete two-step system treatment for acne breakouts. This unique acne treatment can also help to reduce the appearance of scars without over-drying the skin. It has been formulated to address scarring associated with acne problems while also providing a treatment. It will also be appreciated that, other acne treatments typically recommend more than two-steps for complete results.

Typical acne treatments require: a wash (which can dry out the skin); a toner (which can be used to repair the skin from damage caused by the wash); a treatment for treating the acne; and a concealer for hiding the treatment. Whereas an embodiment of the presently disclosed acne treatment is two-step treatment regimen comprising a wash and a cream treatment, while also assisting in scar reduction.

In this embodiment, a two-step treatment regimen comprises an Acne Rescue Wash and an Acne Rescue Cream. This can provide a complete treatment through a four-way mode of action compared to other products that provide partial treatment and require additional steps for a complete treatment. In providing a complete treatment, this two-step treatment combines the following actions:
Anti-microbial;
Balance excessive sebum secretion;
Exfoliation action; and
Reduce inflammation.

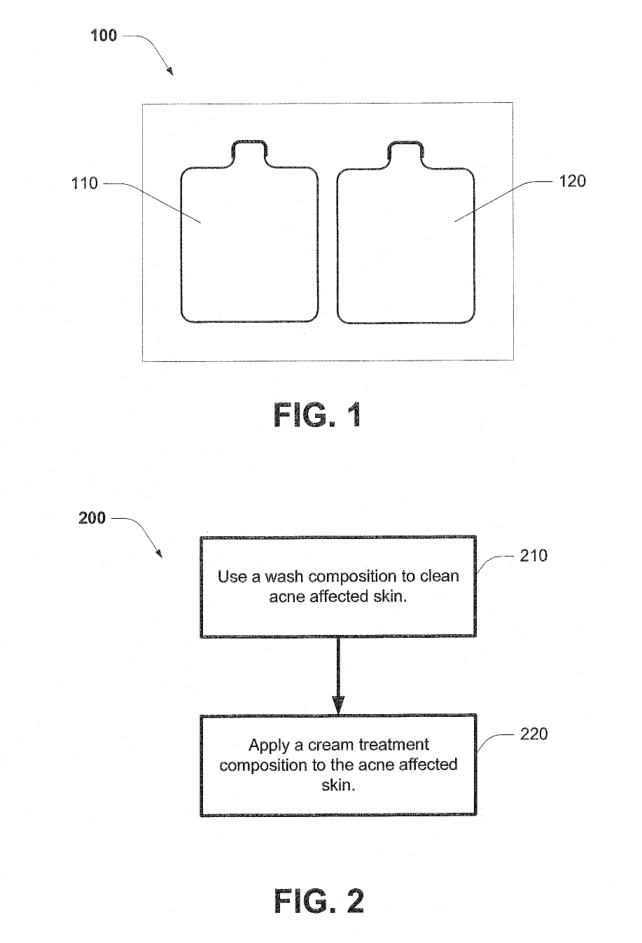

FIG. 1 shows an example kit 100 for providing a two-step acne treatment regimen. The kit comprises: a container 110 of wash composition (or Acne Rescue Wash); and a container 120 of cream treatment composition (or Acne Rescue Cream).

FIG. 2 shows an example flowchart 200 of a method for a two-step acne treatment regimen. The method comprising the steps of:
STEP 210, using a wash composition to cleans acne affected skin of an individual; and
STEP 220, applying a cream treatment composition to the acne affected skin.

In an embodiment, appearance of scars can also be reduced. The Acne Rescue Cream contains Rosehip oil, which assists in the prevention of scar formation. It has been identified that Rosehip oil is a tissue regenerator and can reduce scar tissue on the skin. Rose hip oil also reduces the redness or hyper-pigmentation, reducing the formation of keloid scar tissue formed and loosening up fibrous cords.

In an embodiment, the Acne Rescue Wash has a cleansing effect without over-dry the skin (no additional toner is required). This wash is free of salicylic acid that dries out the skin, and free of benzoyl peroxide which is a potential skin irritant. The Acne Rescue Wash consists of naturally derived mild cleansers derived from Palm Oil and Coconut Oil) to gently cleanse affected area without further irritating the skin.

It will be appreciated that other benefits of this treatment includes; it does not contain any Comedogenic ingredients that block pores during treatment. Many common Acne treatments contain. Cetearyl alcohol, Isopropyl Palmitate and Sodium Lauryl Sulfate (SLS).

The Acne Rescue Kit comprises an Acne Rescue Wash and an Acne Rescue Cream, and can provide a treatment specifically formulated to:
reducing blackheads, whiteheads and redness;
reducing Inflammation;
reducing and clearing-out congested pores and dead cells;
reducing oiliness;
reducing appearance of scars;
keeping skin soft and smooth; and
killing acne-causing bacteria.

In an embodiment, each formulation contains a synergistic blend of ingredients including: homeopathic ingredients, essential oils and natural derivatives. The functions of these ingredients can be described as:
Lactic Acid, gently exfoliates and unblocks clogged pores;
Zinc PCA, enables gentle absorption of excessive sebum and assists in regulating overall production of sebum in the skin, as excessive sebum production can contribute to blocked-pores;
Lauryl Glucoside (derived from Palm Oil) gently cleanses affected area without further irritating the skin;
Cocamidopropyl Betaine (derived from Coconut Oil) gently cleanses affected area without further irritating the skin;
Tea Tree Oil, which contains a constituent known as Terpinen-4-Ol, assists with the control of antimicrobial activity, and in particular kills Propionibacterium Acnes (bacteria that encourages the growth of Acne on the skin) in acne lesions;
Witch Hazel acts as an antiseptic that assists with cleansing the skin and killing acne-causing bacteria;
Calendula Extract can have an anti-inflammatory effect;
Chamomile Extracts—including various isolated constituents within Chamomile—can have an anti-inflammatory effect;
Aloe Vera acts as a moisturizer to revitalize and restore the lost moisture of the skin, and assists with soothing inflamed skin conditions and rebuilding scarred damaged skin areas;
Palmarosa Oil delivers exceptional hydration to the skin, and can assist in renewing of skin cells and the regulation of sebum production; and
Rosehip Oil assists in minimising the appearance scars.

In an example embodiment, active ingredients can include: witch hazel distillate, Zn PCA, Lactic acid, Tea Tree Oil and Rose hip oil.

In an example embodiment, an Acne Rescue Wash can comprise:
Melaleuca Alternifolia (Tea Tree) Oil; and
Hamamelis Virginiana (Witch Hazel) Distillate.

This Acne Rescue Wash also typically comprises:
Lavandula Angustifolia (Lavender) Oil; and/or
Calendula Officinalis (Calendula) Extract 1:2.

In this example, the Acne Rescue Wash can also comprise any one or more of the following ingredients:
Aloe Vera Inner Leaf Juice Powder 200:1;
Sodium Chloride (Natrum Muriaticum) 12×;
Silica 3×; and
Palmarosa Essential Oil.

In this example, the Acne Rescue Wash can further comprise (typically as a filler) any one or more of the following ingredients:
Disodium Cocoamphodiacetate;
Caprylyl Glycol (and) Sorbic Acid (and) Phenoxyethanol;
Benzyl Alcohol;
PEG-150 Distearate;
Polysorbate 20;
Phenoxyethanol;
Lactic Acid;
Cocamidopropyl Betaine;
Lauryl Glucoside; and
Purified Water.

In an example embodiment, an Acne Rescue Cream can comprise:
Melaleuca Alternifolia (Tea Tree) Oil;
Hamamelis Virginiana (Witch. Hazel) Distillate; and
Zinc PCA.

This Acne Rescue Cream also typically comprises:
Rosa Canina Fruit (Rose Hip) Oil; and/or
Garcinia Mangostana (Mangosteen) Extract.

In this example, the Acne Rescue Cream can also comprise any one or more of the following ingredients:
Calcarea Carbonica (Calcium Carbonate) 3×
Pulsatilla Vulgaris 3×;
Calendula Officinalis 6×;
Lactic Acid; and
Chamomilla Recutita (Chamomile) Extract.

In this example, the Acne Rescue Wash can further comprise (typically as a filler) any one or more of the following ingredients:
Glyceryl Stearate;
Cetyl Palmitate (and) Sorbitan Palmitate (and) Sorbitan Olivate (Oliwax);
Caprylyl Glycol (and) Sorbic Acid (and) Phenoxyethanol (Optiphen Plus);
Hydroxyethyl Acrylate/Sodium Acryloyldimethyltaurate Copolymer (and) Squalane (and) Polysorbate 60 (Simulgel NS);
Stearic Acid;
Glycerine 99.5% (Vege);
Capric/Caprylic Triglycerides; and
Purified water.

It will be appreciated that although oils have been used in skin therapies, a blend of oils can provide a synergistic result not obtained by application of any one individual component at the set constituent concentration.

By way of example, a synergistic blend can be obtained from the following active ingredients, in a CCTG base with Squalene as penetration enhancer;
Rose hip oil;
Lavender oil;
Calendula oil;
Evening primrose oil;
Coconut oil;
Avocado oil;
Olive oil;
Arnica oil;
Hypericum oil; and
Everlasting oil.

This synergistic blend, as detailed below, can assist in reducing the effect of scaring, stretch marks and uneven tone; which none of the individual ingredient is expected to produce at that specified concentration.

| Ingredient & percentage | Description |
|---|---|
| Rose hip oil 0.5% | Pure Rosehip oil is typically used to reduce |
| | scarring at 100% concentration. No evidence |
| | for its scar reducing effect when applied at |
| | 0.5% concentration. |
| Lavender 0.5% | Lavender is typically used to reduce |
| | inflammatory skin conditions, and at 100% |
| | concentration as pure lavender oil may show |
| | some scar reduction effect. No evidence of |
| | any such effect when applied at 0.5% |
| | concentration. |
| Calendula oil 0.05% | Used mainly in inflammatory skin disorder, |
| | no specific use in scar, stretch mark and |
| | uneven skin tone reduction. |
| Evening primrose | Topically used in inflammatory skin |
| oil 0.2% | disorders. No specific use in scar, stretch |
| | mark and uneven skin tone reduction. Increase |
| | fluidity and flexibility of the sin. |
| Coconut oil 2% | Carrier oil only, no use in scar, stretch mark |
| | and uneven skin tone reduction |
| Avocado oil 2% | Traditionally used for its healing and anti- |
| | wrinkle property. No specific use in scar, |
| | stretch mark and uneven skin tone reduction. |
| Olive oil 0.075% | Carrier oil, No specific use in scar, stretch |
| | mark and uneven skin tone reduction. |
| Arnica oil 0.25% | Soothing, traditionally used for its analgesic |
| | and antiseptic action. No specific use in scar, |
| | stretch mark and uneven skin tone reduction. |
| Hypericum healing | Topically for mild burns, bruises and many |
| oil 0.15% | skin conditions. No specific use in scar, |
| | stretch mark and uneven skin tone reduction. |
| Everlasting oil | Topically anti-inflammatory action. No |
| 0.0025% | specific use in scar, stretch mark and uneven |
| | skin tone reduction. |

In review, none of the individual ingredient is expected to reduce the effect of scaring, stretch marks and uneven tone at the specified concentration, whereas this synergistic blend can assist in these matters. This is summarised in the table below, where ‘x’ denotes no effect and ‘✓’ denotes an observable effect.

| | | Scar | Stretch mark | Uneven skin |
|---|---|---|---|---|
| | | reducing | reducing | tone reducing |
| Ingredient | Concentration | effect | effect | effect |
| Rose hip oil | 0.5% | X | X | X |
| Lavender | 0.5% | X | X | X |
| Calendula oil | 0.05% | X | X | X |
| Evening | 0.2% | X | X | X |
| primrose oil | | | | |
| Coconut oil | 2% | X | X | X |
| Avocado oil | 2% | X | X | X |
| Olive oil | 0.075% | X | X | X |
| Arnica oil | 0.25% | X | X | X |
| Hypericum | 0.15% | X | X | X |
| healing oil | | | | |
| Everlasting | 0.0025% | X | X | X |
| oil | | | | |
| Synergistic | Combined at | ✓ | ✓ | ✓ |
| blend of all | the above | | | |
| the above. | concentrations | | | |

In an embodiment, by way of example only, an Acne Rescue Wash comprises:
Sodium Chloride (Natrum Muriaticum) 12×, 0.050% w/w;
Silica 3×, 0.050% w/w;
Cocoamidopropyl Betaine, 4.000% w/w;
PEG 150 Distearate, 4.000% w/w;
Polysorbate 20, 4.000% w/w;
Di Sodium Cocoamphodiacetate, 2.000% w/w;
Lauryl Glucoside, 2.000% w/w;
Optiphen Plus (Caprylyl Glycol, Sorbic acid, Phenoxyethanol), 1.5000% w/w;
Witchazel Distillate, 1.000% w/w;
Calendula Officinalis Extract 1:2, 0.500% w/w;
Phenoxyethanol, 0.500% w/w;
Benzyl Alcohol, 0.500% w/w;
Lavender (Lavandula Angustifolia) Oil, 0.400% w/w;
Tea Tree Oil, 0.400% w/w;
Palmarosa Essential Oil, 0.200% w/w;
Lactic Acid, 0.200% w/w;
Aloe Vera Inner Leaf Juice Powder 200:1, 0.050% w/w; and
Purified Water, to 100.000% w/w.

In an embodiment, by way of example only, an Acne Rescue Cream comprises:
Calendula Officinalis 6×, 1.000% w/w;
Calcium Carbonate 3×, 0.500% w/w;
Pulsatilla Vulgaris 3×, 0.500% w/w;
Capric/Caprylic acid Triglycerides, 8.000% w/w;
Oliwax LC, 4.000% w/w;
Stearic Acid, 4.000% w/w;
Lactic Acid, 2.000% w/w;
Simugel NS (Hydroxyethyl Acrylate/Sodium Acryloydimethyltaurate copolymer, Squalene and Polysorbate 60), 2.000% w/w;
Witchazel Distillate, 2.000% w/w;
Zinc PCA, 2.000% w/w;
Sodium Hydroxide, 46% solution, 1.700% w/w;
Optiphen Plus (Caprylyl Glycol, Sorbic acid, Phenoxyethanol), 1.500% w/w;
Glycerine 99.5% (Vege), 1.000% w/w;
Rosehip oil, 1.000% w/w;
Glyceryl Stearate, 1.000% w/w;
Tea Tree Oil, 0.900% w/w;
Chamomile Extract 1:1, 0.500% w/w;
Mangosteen Extract, 0.500% w/w; and
Purified water, to 100.000% w/w.

It will be appreciated that the disclosed kit and method provides a treatment of acne.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprises, comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

The claims defining the invention are as follows:

1. A kit for a two-step acne treatment regimen, the kit comprising:
a wash composition; and
a cream treatment composition.

2. The kit according to claim 1, wherein the treatment regimen further assists in scar reduction.

3. The kit according to claim 1, wherein the wash composition comprises:
Melaleuca Alternifolia (Tea Tree) Oil; and
Hamamelis Virginiana (Witch Hazel) Distillate.

4. The kit according to claim 3, wherein the wash composition comprises any one or more of the following:
Lavandula Angustifolia (Lavender) Oil; and
Calendula Officinalis (Calendula) Extract 1:2.

5. The kit according to claim 4, wherein the wash composition comprises any one or more of the following:
Aloe Vera Inner Leaf Juice Powder 200:1;
Sodium Chloride (Natrum Muriaticum) 12×;
Silica 3×; and
Palmarosa Essential Oil.

6. The kit according to claim 5, wherein the wash composition comprises any one or more of the following:
Disodium Cocoamphodiacetate;
Caprylyl Glycol (and) Sorbic Acid (and) Phenoxyethanol;
Benzyl Alcohol;
PEG-150 Distearate;
Polysorbate 20;
Phenoxyethanol;
Lactic Acid;
Cocamidopropyl Betaine;
Lauryl Glucoside; and
Purified Water.

7. The kit according to claim 1, wherein the cream treatment composition comprises:
Melaleuca Alternifolia (Tea Tree) Oil;
Hamamelis Virginiana (Witch Hazel) Distillate; and
Zinc PCA.

8. The kit according to claim 7, wherein the cream treatment composition comprises any one or more of the following:
Rosa Canina Fruit (Rose Hip) Oil; and
Garcinia Mangostana (Mangosteen) Extract.

9. The kit according to claim 8, wherein the cream treatment composition comprises any one or more of the following:
Calcarea Carbonica (Calcium Carbonate) 3×
Pulsatilla Vulgaris 3×;
Calendula Officinalis 6×;
Lactic Acid; and
Chamomilla Recutita (Chamomile) Extract.

10. The kit according to claim 9, wherein the cream treatment composition comprises any one or more of the following:
Glyceryl Stearate;
Cetyl Palmitate (and) Sorbitan Palmitate (and) Sorbitan Olivate (Oliwax);
Caprylyl Glycol (and) Sorbic Acid (and) Phenoxyethanol (Optiphen Plus);
Hydroxyethyl Acrylate/Sodium Acryloyldimethyltaurate Copolymer (and) Squalane (and) Polysorbate 60 (Simulgel NS);
Stearic Acid;
Glycerine 99.5% (Vege);
Capric/Caprylic Triglycerides; and
Purified water.

11. The kit according to claim 1, wherein the wash composition comprises:
Sodium Chloride (Natrum Muriaticum) 12× at 0.050% w/w; Silica 3× at 0.050% w/w; Cocoamidopropyl Betaine at 4.000% w/w; PEG 150 Distearate at 4.000% w/w; Polysorbate 20 at 4.000% w/w; Di Sodium Cocoamphodiacetate at 2.000% w/w; Lauryl Glucoside at 2.000% w/w; Optiphen Plus (Caprylyl Glycol, Sorbic acid, Phenoxyethanol) at 1.5000% w/w; Witchazel Distillate at 1.000% w/w; Calendula Officinalis Extract 1:2 at 0.500% w/w; Phenoxyethanol at 0.500% w/w; Benzyl Alcohol at 0.500% w/w; Lavender (Lavandula Angustifolia) Oil at 0.400% w/w; Tea Tree Oil at 0.400% w/w; Palmarosa Essential Oil at 0.200% w/w; Lactic Acid at 0.200% w/w; Aloe Vera Inner Leaf Juice Powder 200:1 at 0.050% w/w; and Purified Water to 100.000% w/w.

12. The kit according to claim 1, wherein the cream treatment composition comprises: Calendula Officinalis 6× at 1.000% w/w; Calcium Carbonate 3× at 0.500% w/w; Pulsatilla Vulgaris 3× at 0.500% w/w; Capric/Caprylic acid Triglycerides at 8.000% w/w; Oliwax LC at 4.000% w/w; Stearic Acid at 4.000% w/w; Lactic Acid at 2.000% w/w; Simugel NS (Hydroxyethyl Acrylate/Sodium Acryloydimethyltaurate copolymer, Squalene and Polysorbate 60) at 2.000% w/w; Witchazel Distillate at 2.000% w/w; Zinc PCA at 2.000% w/w; Sodium Hydroxide 46% solution at 1.700% w/w; Optiphen Plus (Caprylyl Glycol, Sorbic acid, Phenoxyethanol) at 1.500% w/w; Glycerine 99.5% (Vege) at 1.000% w/w; Rosehip oil at 1.000% w/w; Glyceryl Stearate at 1.000% w/w; Tea Tree Oil at 0.900% w/w; Chamomile Extract 1:1 at 0.500% w/w; Mangosteen Extract at 0.500% w/w; and Purified water to 100.000% w/w.

13. The kit according to claim 1, wherein the treatment regimen further assists in scar reduction, and the cream treatment composition includes:
Rose hip oil; Lavender oil; Calendula oil; Evening primrose oil; Coconut oil; Avocado oil; Olive oil; Arnica oil; Hypericum oil; and Everlasting oil.

14. The kit according to claim 13, wherein the treatment regimen further assists in scar reduction, and the cream treatment composition including:
substantially 0.5% Rose hip oil;
substantially 0.5% Lavender oil;
substantially 0.05% Calendula oil;
substantially 0.2% Evening primrose oil;
substantially 2% Coconut oil;
substantially 2% Avocado oil;
substantially 0.075% Olive oil;
substantially 0.25% Arnica oil;
substantially 0.15% Hypericum healing oil; and
substantially 0.0025% Everlasting oil.

15. A kit for a two-step acne treatment regimen, the kit comprising:
a wash composition; and
a cream treatment composition;
wherein the cream treatment composition comprises: Melaleuca Alternifolia (Tea Tree) Oil; Hamamelis Virginiana (Witch Hazel) Distillate; Zinc PCA; Rosa Canina Fruit (Rose Hip) Oil; and Garcinia Mangostana (Mangosteen) Extract.

16. A method for a two-step acne treatment regimen, the method comprising the steps of:
a) using a wash composition to clean acne affected skin of an individual; and
b) applying a cream treatment composition to the acne affected skin.

17. The method according to claim 16, wherein the treatment regimen further assists in scar reduction.

18. The method according to claim 16, wherein the wash composition comprises:
Melaleuca Alternifolia (Tea Tree) Oil;
Hamamelis Virginiana (Witch Hazel) Distillate; and
Lavandula Angustifolia (Lavender) Oil; and
Calendula Officinalis (Calendula) Extract 1:2.

19. The method according to claim 18, wherein the cream treatment composition comprises:
Melaleuca Alternifolia (Tea Tree) Oil;
Hamamelis Virginiana (Witch Hazel) Distillate; and
Zinc PCA.

20. The method according to claim 19, wherein the cream treatment composition comprises:
Rosa Canina Fruit (Rose Hip) Oil; and
Garcinia Mangostana (Mangosteen) Extract.
